(19) United States
(12) Patent Application Publication
(10) Pub. No.: US 2022/0251552 A1
(43) Pub. Date: Aug. 11, 2022

(54) Regenerative Therapy Based on miRNA-302 Mimics for Enhancing Host Recovery from Pneumonia Caused by Streptococcus pneumoniae

(71) Applicants: The Trustees of the University of Pennsylvania, Philadelphia, PA (US); Temple University - Of The Commonwealth System Of Higher Education, Philadelphia, PA (US)

(72) Inventors: Hao SHEN, Philadelphia, PA (US); Yan WANG, Hamilton, NJ (US); Ying TIAN, Philadelphia, PA (US)

(21) Appl. No.: 17/441,596

(22) PCT Filed: Mar. 23, 2020
(86) PCT No.: PCT/US2020/024206

§ 371 Date: Sep. 21, 2021

Related U.S. Application Data
(60) Provisional application No. 62/823,400, filed on Mar. 25, 2019.

(51) Int. Cl. C12N 15/113; A61P 11/00; A61P 31/04

(52) U.S. Cl. CPC C12N 15/113; A61P 11/00; A61K 48/00; C12N 2310/141; A61P 31/04

(57) ABSTRACT

The present invention relates to methods and compositions comprising a miR-302 mimic/s for treatment of lung injury. The miRNA-302 mimic/s facilitate host recovery from lung injury caused due to, for example, bacterial pneumonia

### CROSS-REFERENCE TO RELATED APPLICATIONS

The present application claims priority under 35 U.S.C. § 119(e) to U.S. Provisional Patent Application No. 62/823,400 filed, Mar. 25, 2019, which is incorporated herein by reference in its entirety.

### STATEMENT REGARDING FEDERALLY SPONSORED RESEARCH OR DEVELOPMENT

This invention was made with government support under Grant Nos. R01 HL 132115-01 and R21 AI 128569-01A1 awarded by the National Institutes of Health (NIH). The government has certain rights in the invention.

### BACKGROUND OF THE INVENTION

Pneumonia is a major cause of severe morbidity, often resulting in hospitalization, admission to intensive care units, long recovery, and high rate of mortality. Streptococcus pneumoniae (Sp) is the leading cause of bacterial pneumonia and secondary pneumonia following influenza virus infection. The pathobiology of pneumonia is characterized by robust host immune responses that causes lung damage. Studies of microbial infection have mostly focused on bacterial virulence and host immune responses, with the goal of developing interventions based on antimicrobials or vaccines. However, full recovery from bacterial pneumonia is dependent not only on clearance of microbial pathogens but also on regeneration of the damaged airway epithelium. Failure to repair epithelial damage can disrupt the epithelial barrier that protects the lung from external insults, leading to susceptibility to recurring infections and development of chronic and progressive lung diseases, which includes chronic obstructive pulmonary disease (COPD), idiopathic pulmonary fibrosis (IPF), and emphysema.

It is known that the regulatory pathways involved in tissue growth and differentiation during embryogenesis are reactivated in the process of regeneration following injury in adults. Therefore, microRNAs (miRs) have emerged as key modulators of regeneration process by controlling expression of signaling and transcription factors involved in multiple facets of tissue development and regeneration. Despite the knowledge that miRs can modulate tissue regeneration, the role of miRs in epithelial regeneration as well as repair and recovery from lung injuries is largely unexplored.

There remains a need in the art for methods and compositions for treating lung injuries using miRs as therapeutic agents. The present invention addresses this need.

### SUMMARY OF THE INVENTION

In one aspect, the invention provides a method of treating a lung injury in a subject in need thereof, the method comprising administering to the subject a therapeutically effective amount of at least one miR-302 mimic.

In another aspect, the invention provides a method of regeneration of alveolar epithelial cells I (AEC) and alveolar epithelial cells II (AECII) in lungs of a subject, the method comprising administering to the subject a composition comprising a therapeutically effective amount of at least one miR-302 mimic.

In yet another aspect, the invention provides a kit comprising a composition comprising at least one miR-302 mimic, and an instructional material for use thereof, wherein the instructional material comprises instructions for treating a lung injury in a subject, wherein treating comprises administering the at least one miR-302 mimic to the subject.

In certain embodiments, the at least one miR-302 mimic comprises miR-302b mimic or miR-302c mimic.

In certain embodiments, administering the at least one miR-302 mimic promotes regeneration of alveolar epithelial cells I (AECI) and alveolar epithelial cells II (AEC II) in a lung of the subject.

In certain embodiments, administering the at least one miR-302 mimic results in upregulation of expression of at least one cell proliferation gene in a lung of the subject. In certain embodiments, the at least one cell proliferation gene is selected from the group consisting of Ccnd1, Ccnd2, Ctgf, Cyr61, Nusap1, Myh10, Cks2 and Brca2.

The method of claim 1, wherein administering the at least one miR-302 mimic results in downregulation of expression of Cdkn1a gene.

In certain embodiments, the lung injury is caused by a bacterial infection. In certain embodiments, the bacterial infection is bacterial pneumonia.

In certain embodiments, the at least one miR-302 mimic is administered intravenously.

In certain embodiments, the at least one miR-302 mimic further comprises a pharmaceutically acceptable carrier or adjuvant.

In certain embodiments, the at least one miR-302 mimic is mammalian. In certain embodiments, the at least one miR-302 mimic is human.

In certain embodiments, the at least one miR-302 mimic is engineered.

In certain embodiments, the subject is mammal. In certain embodiments, the mammal is human.

### BRIEF DESCRIPTION OF THE FIGURES

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1G show bacterial clearance and alveolar epithelial damage and repair in SpT4-infected mice. Lung tissues were collected on 2, 7, 14, and 30 dpi with SpT4. (FIG. 1A) Hematoxylin and eosin staining. (FIG. 1B) Immunostaining with antibodies to the type 4 capsular of SpT4. (FIG. 1C) Bacteria loads in lung homogenate measured by CFU plating (LOD: limit of detection). (FIG. 1D and FIG. 1F) Immunostaining with mAb to T1α (FIG. 1D), SPC (FIG. 1F) and CC10 (FIG. 1F). DAPI: (FIGS. 1C, 1D, and 1F). Scale bar: 500 μm (FIG. 1A), 50 μm (FIGS. 1B, 1D and 1F). (FIG. 1E and FIG. 1G) qRT-PCR of T1α and SPC mRNA for in lung tissue. Data are mean±SEM. *P<0.05, **P<0.01, ***P<0.005 versus non-infected normal lungs (0 dpi) (Student's t test).

FIGS. 2A-2B show miR-302 expression in AEC after SpT4 infection. (FIG. 2A) qRT-PCR analysis of miR-302-367 polycistron (miR-302b/c/a/d family and miR-367) from isolated lung epithelial cells of mouse distal lung at 0, 2, 7, 14, 30 dpi. (FIG. 2B) In situ hybridization of digoxigenin-labeled LNA miR-302c antisense probe and LNA-scrambled control probe (Exiqon) on lung tissue sections at 0 and 7 dpi. Arrows point to the alveolar epithelial cells. Scale bars: 50 μm. Data were analyzed using Student's t test (n=4 per group). Data shown are means±SEM *P<0.05, **P<0.01.

FIGS. 3A-3I illustrate effects of miR-302b/c mimics treatment on SpT4-infected mice. (FIG. 3A) Schematic of experimental design. Mice were with SpT4 on day 0, then treated with either miR-302b/c or negative control (Ctrl) mimics at 5 &6 dpi. and monitored daily for survival (FIG. 3B), gain of body weight (FIG. 3C), and blood oxygen levels (FIG. 3F). Total protein levels (FIG. 3D) and LDH activities (FIG. 3E) in BALF at indicated dpi. Pulmonary functions (FIG. 3G) were analyzed on 21 dpi for compliance, forced expiratory volume in 0.1 seconds (FEV0.1), and forced vital capacity (FVC). (FIG. 3H) Immunostaining of lung sections with mAb to T1α and to SPC. Scale bar, 50 μm. (FIG. 3I) qRT-PCR of T1α and SPC mRNA in the lung tissues. Normal: uninfected/untreated control. Data in FIG. 3B) was analyzed using Gehan-Breslow-Wilcoxon test of cumulative data (n=14 for miR-302b/c, n=16 for Ctrl mimics). Data shown are means±SEM (FIGS. 3C-3G, n=4 per group; FIG. 3I, n=10 per group). *P<0.05, **P<0.01, ***P<0.001.

FIGS. 4A-4D show AEC proliferation following miR-302b/c mimics treatment of SpT4-infected mice. (FIG. 4A) Schematic of experimental design. (FIG. 4B and FIG. 4C) Confocal images of lung sections at 7 dpi by Click-iT EdU Alexa Fluor 488 imaging and co-immunostaining with mAb to SPC (AECII, FIG. 4B) and to Hopx (AECI, FIG. 4C), and quantification of EdU⁺SPC⁺ and EdU⁺Hopx⁺ cells as % of total SPC⁺ and Hopx⁺ cells, respectively. Arrows in (FIGS. 4B and 4C) point to nucleus of proliferating (EdU⁺) cells. Quantitative analyses represent counting of multiple fields from 5 independent samples per group (˜2200 SPC⁺ cells and ˜765 Hopx⁺ cell per sample). Data are means±SEM (n=5-6 per group). P value was calculated using Student's t test. (FIG. 4D) Expression of indicated genes of interest by qRT-PCR analysis of the mRNA of isolated lung epithelial cells at 7, 14, and 21 dpi. Data are means±SEM (n=3 per group and time) *P<0.05, **P<0.01, ***P<0.001 (Student's t test). Scale bar, 10 μm.

FIGS. 5A-5B illustrate tissue damage and resolution in Sp-infected mouse lung. (FIG. 5A) Histological analysis of lung sections at 7 dpi using indicated antibodies with nuclear counterstain (DAPI). P63 (basal cells), β-tubulin IV (ciliated cells), αSMA (perivascular and peribronchiolar smooth muscle, and fibroblasts at the alveolar entrance ring), PECAM1 (vascular endothelial cells), respectively. (FIG. 5B) Masson's trichrome stained lung sections at 0, 2, 7, 14, 30 dpi. Scale bar, 50 μm.

FIGS. 6A-6B show expression of miR-302b/c mimics in the lung and tissue histology after systemic treatment with mimics. (FIG. 6A) Experimental design and qRT-PCR showing miR-302b/miR-302c levels in the lung at various time points after intravenous (i.v.) treatment of mimics. (FIG. 6B) Masson's trichrome staining showing fibrotic tissue formation and resolution from lungs with control mimics (Ctrl mimics)- or miR-302b/c mimics (miR-302b/c)-treated mice at indicated dpi (n=3 per group and time point). Scale bar, 50 μm.

FIGS. 7A-7E depict effects on cell apoptosis in Sp-infected lung with miR-302b/c mimics treatment. (FIG. 7A) Histological analysis and (FIG. 7B) quantification of apoptotic cells measured by TUNEL staining at indicated dpi. Quantification data are means±SEM (n=10 per group and time point). (FIG. 7C) Representative flow cytometry plots assessing cleaved Caspase-3 expressions from Epcam⁺ cells in lung with control mimics- or miR-302b/c-treated mice at 7 dpi. (FIG. 7D) Group data quantify number of Epcam⁺Caspase-3⁺ cells (n=3 per group and time point). (FIG. 7E) qRT-PCR analysis of Dapk1, Stk17b, Bax at the indicated dpi from isolated lung epithelial cells. Data are means±SEM (n=10 per group) *P<0.05 versus control mimic treated lungs (Student's t test). Scale bar, 50 μm.

FIGS. 8A-8J show cell proliferation in Sp-infected lung with miR-302b/c mimics treatment. Histological analysis and quantification of lung sections showing proliferating (EdU) (FIG. 8A) basal cells (anti-p63 (FIG. 8B) ciliated cells (anti-β-Tubulin IV), (FIG. 8C and FIG. 8D) club cells (anti-CC10), (FIG. 8E and FIG. 8F) smooth muscle cells (anti-αSMA), (FIG. 8G and FIG. 8H) macrophages (anti-F4/80), (FIG. 8I and FIG. 8J) endothelial cells (anti-PECAM1). Arrowhead points to nucleus of cell type specific proliferating cells. Scale bar, 50 μm. Data are means±SEM (FIG. 8D, FIG. 8F, FIG. 8H, FIG. 8J: n=3 per group). *P<0.05 versus control mimic-treated lungs using Student's t test. FIG. 8A, FIG. 8B, FIG. 8C, FIG. 8E, FIG. 8G, FIG. 8I: left, un-infected normal lung; middle, control mimics-treated lung at 7 dpi; right, miR-302b/c-treated lung at 7 dpi.

### DETAILED DESCRIPTION OF THE INVENTION

This invention is based on studies conducted on a murine pneumonia model with diffuse bacterial infection of alveolar spaces, resulting in acute inflammation and substantial damage to alveolar epithelial cells (AEC), followed by a slow process of regeneration over an extended period (>30 days). In the murine model, the expression of miR-302 was up-regulated in AEC and coincided with AEC regeneration. Without wishing to be bound by any particular theory, the transient expression of miR-302 genes critical for fetal lung development stimulate local stem cells in the lung parenchyma to regenerate epithelium and promote animal recovery following Streptococcus pneumoniae (Sp) infection. The present invention establishes that the treatment of Sp-infected mice with miR-302 mimics improved lung function, host recovery, and survival by promoting proliferation of alveolar epithelial progenitor cells to regenerate AEC and repair damaged alveolar epithelium. These findings suggest that miRNA-based therapy is useful as a powerful therapeutic to promote AEC regeneration and enhance host recovery from bacterial pneumonia.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

By the term “modified” as used herein, is meant a changed state or structure of a molecule. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

As use herein, the term “miRNA” comprises micro RNA which is about 15 nt to about 50 nt in length.

As used herein, the term “miR-302 mimic” encompasses a miR-302 duplex, a chemically modified double stranded miR-302, an unmodified double stranded miR-302, a single stranded chemically modified miR-302 or a single stranded unmodified miR-302.

The term “oligonucleotide” typically refers to short polynucleotides, generally no greater than about 60 nucleotides. It will be understood that when a nucleotide sequence is represented by a DNA sequence (i.e., A, T, G, C and optionally, modified bases), this also includes an RNA sequence (i.e., A, U, G, C and optionally, modified bases) in which “U” replaces “T.”

As used herein, “Parenteral” administration of miR-302 mimics includes, e.g., subcutaneous (s.c.), intravenous (i.v.), intramuscular (i.m.), or intrasternal injection, or infusion techniques.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

“Pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutical composition” or “pharmaceutically acceptable composition” refers to a mixture of at least one compound or molecule useful within the invention with a pharmaceutically acceptable carrier. The pharmaceutical composition facilitates administration of the compound or molecule to a patient. Multiple techniques of administering a compound or molecule exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound or molecule useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “protein”, “peptide”, and “polypeptide” are used interchangeably, and refer to a compound comprised of amino acid residues covalently linked by peptide bonds. The term “peptide bond” means a covalent amide linkage formed by loss of a molecule of water between the carboxyl group of one amino acid and the amino group of a second amino acid. A protein or peptide must contain at least two amino acids, and no limitation is placed on the maximum number of amino acids that may comprise the sequence of a protein or peptide. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Proteins” include, for example, biologically active fragments, substantially homologous proteins, oligopeptides, homodimers, heterodimers, variants of proteins, modified proteins, derivatives, analogs, and fusion proteins, among others. The proteins include natural proteins, recombinant proteins, synthetic proteins, or a combination thereof. A protein may be a receptor or a non-receptor.

The term “treat,” as used herein, means reducing the frequency with which symptoms are experienced by a subject or administering an agent or compound to reduce the frequency and/or severity with which symptoms are experienced. As used herein, “alleviate” is used interchangeably with the term “treat.”

As used herein, “treating a disease, disorder or condition” means reducing the frequency or severity with which a symptom of the disease, disorder or condition is experienced by a subject. Treating a disease, disorder or condition may or may not include complete eradication or elimination of the symptom.

Ranges throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Methods

In one aspect, the invention provides a method of treating a lung injury in a subject in need thereof, the method comprises administering to the subject a therapeutically effective amount of at least one miR-302 mimic.

In certain embodiments, the miR-302 mimic comprises miR-302b mimic or miR-302c mimic. The miR-302b and miR-302c belong to a cluster miR-302-367, known as the miR-302 family, which is highly expressed at early stages of fetal mouse lung development and contributes to enhanced proliferation of lung progenitor cells during embryogenesis.

In certain embodiments, administering the at least one miR-302 mimic promotes regeneration of alveolar epithelial cells I (AECI) and alveolar epithelial cells II (AEC II) in a lung of the subject. Typically, AECI comprise the major gas exchange surface of the alveolus and are integral to the maintenance of the permeability barrier function of the alveolar membrane whereas AECII are the progenitors of AECI and are responsible for surfactant production and homeostasis

In certain embodiments, administering the miR-302 mimic results in upregulation of expression of at least one cell proliferation gene in a lung of the subject. In certain embodiments, the at least one cell proliferation gene is selected from the group consisting of Ccnd1, Ccnd2, Ctgf, Cyr61, Nusap1, Myh10, Cks2 and Brca2.

In certain embodiments, administering the miR-302 mimic results in downregulation of expression of Cdkn1a gene.

In certain embodiments, the lung injury is caused by a bacterial infection. In certain embodiments, the bacterial infection is bacterial pneumonia. Pneumonia can occur in both lungs, one lung, or one section of a lung. Pneumococcal disease, which is caused by Streptococcus pneumoniae infection, is a major cause of bacterial pneumonia. Haemophilus influenzae, Chlamydia pneumoniae, Mycoplasma pneumoniae, and Legionella pneumophila are some other major bacteria that cause pneumonia.

In certain embodiments, the miR-302 mimic is administered intravenously. In certain other embodiments, the of miR-302 is administered subcutaneously (s.c.), intramuscularly (i.m.).

In certain embodiments, the miR-302 mimic further comprises a pharmaceutically acceptable carrier or adjuvant such as but not limited to buffers such as neutral buffered saline, phosphate buffered saline and the like; carbohydrates such as glucose, mannose, sucrose or dextrans, mannitol; proteins; polypeptides or amino acids such as glycine;

antioxidants; chelating agents such as EDTA or glutathione; adjuvants (e.g., aluminum hydroxide); and preservatives. Compositions of the present invention are preferably formulated for intravenous administration.

In certain embodiments, the miR-302 mimic is mammalian. In certain embodiments, the miR-302 mimic is human. In certain embodiments, the at least one miR-302 is engineered.

In certain embodiments, the subject is mammal. In certain embodiments, the mammal is human.

In another aspect, the invention provides a method of regeneration of alveolar epithelial cells I (AEC) and alveolar epithelial cells II (AECII) in lungs of a subject, wherein the method comprises administering to the subject a composition comprising a therapeutically effective amount of at least one miR-302 mimic.

In certain embodiments, the composition for regeneration of alveolar epithelial cells I (AEC) and alveolar epithelial cells II (AECII) in lungs of a subject further comprises a pharmaceutically acceptable carrier or adjuvant and at least one miR-302 mimic selected from miR-302b mimic or miR-302c mimic.

### Pharmaceutical Compositions

In one aspect, the invention provides is a composition comprising a miR-302 mimic and a pharmaceutically acceptable carrier or adjuvant. In some embodiments, the miR-302 mimic is duplex, a chemically modified double stranded miR-302, an unmodified double stranded miR-302, a single stranded chemically modified miR-302 or a single stranded unmodified miR-302. In further embodiments, the miR-302 mimic is mammalian. In yet further embodiments, the miR-302 mimic is human. In further embodiments, the miR-302 mimic is engineered.

Pharmaceutical compositions of the present invention may comprise a miR-302 mimic as described herein, in combination with one or more pharmaceutically or physiologically acceptable carriers, diluents, adjuvants or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are preferably formulated for intravenous administration.

Pharmaceutical compositions of the present invention may be administered in a manner appropriate to the disease to be treated (or prevented). The quantity and frequency of administration will be determined by such factors as the condition of the patient, and the type and severity of the patient's disease, although appropriate dosages may be determined by clinical trials.

Pharmaceutical compositions of the present invention may be administered in solid or liquid form such as tablets, capsules, powders, solutions, suspensions, emulsions and the like. Pharmaceutical compositions of the present invention may be administered orally, parenterally, subcutaneously, intravenously, intramuscularly, intraperitoneally, by nasal instillation, by implantation, by intracavitary or intravesical instillation, intraocularly, intraarterially, intralesionally, transdermally, or by the application to mucous membranes. In some embodiments, the composition may be applied to the nose, throat or bronchial tubes, for example by inhalation.

The dosage of the above treatments to be administered to a patient will vary with the precise nature of the condition being treated and the recipient of the treatment. The scaling of dosages for human administration can be performed according to art-accepted practices. The dose for a miR-302 mimic, for example, will generally be in the range 0.01 to about 100 mg for an adult patient, usually administered daily for a period between 1 and 30 days. The preferred daily dose is 0.1 to 10 mg per day although in some instances larger doses of up to 1 mg per day may be used.

Human dosage amounts can initially be determined by extrapolating from the amount of compound used in mice, as a skilled artisan recognizes it is routine in the art to modify the dosage for humans compared to animal models. In certain embodiments it is envisioned that the dosage may include an effective amount from between about 0.001 mg compound/Kg body weight to about 100 mg compound/Kg body weight; or from about 0.05 mg/Kg body weight to about 75 mg/Kg body weight or from about 0.1 mg/Kg body weight to about 50 mg/Kg body weight; or from about 0.5 mg/Kg body weight to about 40 mg/Kg body weight; or from about 0.1 mg/Kg body weight to about 30 mg/Kg body weight; or from about 1 mg/Kg body weight to about 20 mg/Kg body weight. In other embodiments, the effective amount may be about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, or about 100 mg/Kg body weight. In other embodiments, it is envisaged that effective amounts may be in the range of about 2 mg compound to about 100 mg compound. In other embodiments, the effective amount may be about 1, 2, 3, 4, 5, 6, 7, 8, 9, 10, 11, 12, 13, 14, 15, 16, 17, 18, 19, or 20 mg per single dose. In another embodiment, the effective amount comprises less than about 2, 3, 4, 5, 6, 7, 8, 9, 10, 15, 20, 25, 30, 35, 40, 45, 50, 55, 60, 65, 70, 75, 80, 85, 90, 95 mg daily. In an exemplary embodiment, the effective amount comprises less than about 50 mg daily. Of course, the single dosage amount or daily dosage amount may be adjusted upward or downward, as is routinely done in such treatment protocols, depending on the results of the initial clinical trials and the needs of a particular patient.

The precise determination of what would be considered an effective dose is based on factors individual to each subject, including their size, age, sex, weight, and condition of the particular subject. Dosages can be readily ascertained by those skilled in the art from this disclosure and the knowledge in the art.

Optionally, the methods of the invention provide for the administration of a composition of the invention to a suitable animal model to identify the dosage of the composition(s), concentration of components therein and timing of administering the composition(s), which elicit tissue repair, reduce cell death, or induce another desirable biological response. Such determinations do not require undue experimentation, but are routine and can be ascertained without undue experimentation.

The biologically active agents can be conveniently provided to a subject as sterile liquid preparations, e.g., isotonic aqueous solutions, suspensions, emulsions, dispersions, or viscous compositions, which may be buffered to a selected pH. For some applications, liquid formations are desirable because they are convenient to administer, especially by injection. Where prolonged contact with a tissue is desired, a viscous composition may be preferred. Such compositions are formulated within the appropriate viscosity range. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions are prepared by suspending talampanel and/or perampanel in the required amount of the appropriate solvent with various amounts of the other ingredients, as desired. Such compositions may be in admixture with a suitable carrier, diluent, or excipient, such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells or agents present in their conditioned media.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent, such as methylcellulose. Other suitable thickening agents include, for example, xanthan gum, carboxymethyl cellulose, hydroxypropyl cellulose, carbomer, and the like. The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form). Those skilled in the art will recognize that the components of the compositions should be selected to be chemically inert.

### Kit

In one aspect, the invention further provides a kit comprising a composition comprising at least one miR-302 mimic, and an instructional material for use thereof, wherein the instructional material comprises instructions for treating a lung injury in a subject, wherein the treating includes administering the at least one miR-302 mimic to the subject.

In certain embodiments, the composition is as described elsewhere herein. In certain embodiments, the miR-302 mimics are as described elsewhere herein.

It should be understood that the method and compositions that would be useful in the present invention are not limited to the particular formulations set forth in the examples. The following examples are put forth so as to provide those of ordinary skill in the art with a complete disclosure and description of how to make and use the cells, expansion and culture methods, and therapeutic methods of the invention, and are not intended to limit the scope of what the inventor regard as his invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Experimental Examples

The Materials and Methods used in the described experimental examples are set forth herein.

Animals. C57BL/6, CD-1® IGS Mouse mice (6-8 wks old) were purchased from Charles River Laboratories (Horsham, Pa.) and were housed in a specific pathogen-free environment at the animal facilities of the University of Pennsylvania and Temple University. All animal experiments were performed in accordance with Institutional Animal Care and Use Committee approved protocols.

Pathogens and infections. Streptococcus pneumoniae (Sp) strain TIGR4 (serotype 4) were propagated in tryptic soy broth (Difco) at 37° C. and 5% CO₂ without shaking until cultures reached log phase, OD₆₂₀ between 0.8 and 1.0 as determined by Spectronic200 spectrophotometer (Thermo). For lung infection, ˜5×10⁶ colony-forming units (CFU) of TIGR4 in 30 μl PBS was inoculated intranasally (i.n.) to mice that were anaesthetized by intraperitoneal (i.p.) injection with 100 μl Ketamine/Xylazine (100 mg/3.8 mg/kg). The dose was confirmed by viable counting via plating of inoculum after infections. This infection resulted in an acute pneumonia with ˜40% mortality rate; mice either succumbed to infection or cleared bacteria within days. Typically, each experimental group started with 50% more mice than needed, and surviving mice were used for analysis in later time-points (n>3 per time point per group) for injury and repair. Mice were observed for clinical signs of morbidity by monitoring body weights and survival daily. Lung homogenates, bronchoalveolar lavages (BAL) and blood were prepared as described (Wang, Y. et.al, (2017), Mucosal Immunol, 10(1), 250-259), and bacterial titers were determined by serially dilutions and plating in triplicate. The limit of detection (LOD) was 4 CFU/mL of lavage, blood or lung homogenate.

RNA purification and RT-PCR analysis. For qRT-PCR of miR-302-367 cluster concentration in epithelial cells, RNA was extracted from isolated epithelial cells using mirVana miRNA isolation kit (Ambion, Inc.). For gene expression of targeted genes in lung, total RNA was isolated from lung lobe at the indicated days post infection using Trizol reagent, reverse transcribed using High-Capacity cDNA Reverse Transcription Kits (Applied Biosystems). Quantitative real-time PCR (qRT-PCR) was performed with primers as described in Table 1. microRNA qRT-PCR was performed by using the miR-302 LNA PCR primer sets (Exiqon). SYBR green detection of amplification was performed using the StepOne Plus cycler (Applied Biosystems). Transcript expression values were generated with the comparative threshold cycle (Delta CT) method by normalizing to the expression of the GAPDH gene.

TABLE 1
qRT-PCR primer sequences used in this study.

| Gene | | |
|---|---|---|
| name | Forward | Reverse |
| Pdpn | GGTGCCCCCAGGTATAGAAG | ACGCTCTCTCTGCGTTGGTA (SEQ ID |
| (T1a) | (SEQ ID NO. 1) | NO. 2) |
| Sftpc | ACCCTGTGTGGAGAGCTACCA (SEQ ID | TTTGCGGAGGGTCTTTCCT (SEQ ID |
| (SPC) | NO. 3) | NO. 4) |
| Scgblal | ATACCCTCCCACAAGAGACCAGGATA | ACACAGGGCAGTGACAAGGCTTTA (SEQ |
| (CC10) | (SEQ ID NO. 5) | ID NO. 6) |
| Dapk1 | GCTGAACATGGAGCTGACTT (SEQ ID | CAAGGAGGGTCTTGATGACTTC (SEQ |
| | NO. 7) | ID NO. 8) |
| Stk17B | AATCTGCATGAGGTCTACGAAA (SEQ ID | TCGGCTAACTCAGGTAAACAC (SEQ ID |
| | NO. 9) | NO. 10) |
| Bax | GTGGTTGCCCTCTTCTACTTT (SEQ ID | CAGCCCATGATGGTTCTGAT (SEQ ID |
| | NO. 11) | NO. 12) |
| Cdkn1a | TTGTACAAGGAGCCAGGCCAAGAT (SEQ | ACTAAGTGCTTTGACACCCACGGT (SEQ |
| (p21) | ID NO. 13) | ID NO. 14) |
| Ccnd1 | TTCCTCTCCAAAATGCCAGA (SEQ ID | AGGGTGGGTTGGAAATGAAC (SEQ ID |
| (CyclinD1) | NO. 15) | NO. 16) |
| Ccnd2 | GAACCTGGCCGCAGTCACCC (SEQ ID | CGACGGCGGGTACATGGCAA (SEQ ID |
| (CyclinD2) | NO. 17) | NO. 18) |
| Ctgf | AACCGCAAGATCGGAGTG (SEQ ID | TGCTTTGGAAGGACTCACC (SEQ ID |
| | NO. 19) | NO. 20) |
| Cyr61 | TGAGTTAATCGCAATTGGAA (SEQ ID | GTGGTCTGAACGATGCATTTC (SEQ ID |
| | NO. 21) | NO. 22) |
| RacGap1 | CAGATCCAGTGACAATGTTCCA (SEQ ID | TCCACCATCATGAACTGATTCC (SEQ ID |
| | NO. 23) | NO. 24) |
| Nusap1 | GAGGAGGAAGAAGCACAAGAC (SEQ ID | CTACTATCAGTTCCTTTCATCTCCAA |
| | NO. 25) | (SEQ ID NO. 26) |
| Myh10 | GGAATTCGAGAGGCAGAACAA (SEQ ID | AAGGCTCGCTTGGATTTCTC (SEQ ID |
| | NO. 27) | NO. 28) |
| Cks2 | CAGAGTCTAGGATGGGTTCATTAC (SEQ | TCCCAGCTGCACTTCATTT (SEQ ID |
| | ID NO. 29) | NO. 30) |
| Brca2 | ATTTGAACGGCCCAGCAT (SEQ ID | GGCTGGTAAACCTGGAGTAAAG (SEQ |
| | NO. 31) | ID NO. 32) |
| Bc12 | GTGGATGACTGAGTACCTGAAC (SEQ ID | GAGACAGCCAGGAGAAATCAA (SEQ ID |
| | NO. 33) | NO. 34) |
| GAPDH | TGCACCACCAACTGCTTAGC (SEQ ID | GGCATGGACTGTGGTCATGAG (SEQ ID |
| | NO. 35) | NO. 36) |

Histology. Lung tissues were inflated and fixed in 4% paraformaldehyde, embedded in paraffin wax and sectioned at 7 μm intervals. Hematoxylin and Eosin (H&E) staining was performed using standard procedures. Immunohistochemistry was performed using the following antibodies: type 4 pneumococcal capsular polysaccharides (Staten serum institute, 1:500), proSurfactant protein c (SP-C) (Millipore, 1:200), Club cell 10 (CC10) (T-18, Santa Cruz, 1:500), T1α (8.1.1, Hybridoma Bank at University of Iowa, 1:100), Hopx (E-1, Santa Cruz, 1:200), p63 (D2K8x, Cell Signaling Technology, 1:200), β-Tubulin IV (ONS1A6, BioGenex, 1:100), α smooth muscle actin (a SMA) (1A4, Sigma, 1:500), PECAM1 (MEC13.3, BD Pharmingen, 1:100), F4/80 (BM8, eBioscience, 1:250), cleaved caspase-3 (Asp175, 5A1E, cell signaling, 1:400). Slides were mounted with Vectashield mounting medium containing DAPI (Vector Laboratories, Burlingame, Calif., USA). Apoptosis was measured using In Situ Cell Death Detection Kit (Roche). Cell proliferation was measured using Click-iT® EdU (5-ethynyl-2′-deoxyuridine) Alexa Fluor® 488 Imaging Kit (Thermo). The slides were imaged and subjected to an independent blinded analysis, using a Zeiss LSM 710 confocal microscope and ImageJ software. Images shown are representative view of multiple fields from at least five independent samples per group. Quantitation of cell numbers was done using images acquired on confocal microscopy and the ImageJ with the “Cell Counter” plug-in, counting multiple fields from 5 independent samples per group and 2200 SPC⁺ cells and ˜765 Hopx⁺ cell per sample.

Preparation of miRNA mimics and in vivo treatment. miR-302b/c-mimics or standard microRNA mimic negative control was custom-ordered from Dharmacon (GE healthcare), formulated with neutral lipid emulsion (NLE) (BIOO Scientific). To determine the effect of miRNA mimics on respiratory repair after Sp infection, 10-μg NLE-formulated miR-302b/c mimics or a control mimic was administered twice by tail vein injection at 5 and 6 dpi. The sequences of oligonucleotides (oligos) for making miRNA 302 b/c mimics and the miRNA 302 b/c mimics are shown in Table 2.

TABLE 2
Sequences for miRNA 302 b/c mimics

| | miRNA | Mature sequence |
|---|---|---|
| SEQ ID NO. 37 | Oligo for | 5′-UAAGUGCUUCCAUGUUUUAG |
| | miR302b | UAG-3′ |
| | mimic | |
| SEQ ID NO. 38 | Oligo for | 5′-AAGUGCUUCCAUGUUUCAGU |
| | miR302c | GG-3′ |
| | mimic | |
| SEQ ID NO. 39 | miR-106a | CAAAGUGCUAACAGUGCAGGUAG |
| SEQ ID NO. 40 | miR-106b | UAAAGUGCUGACAGUGCAGAU |
| SEQ ID NO. 41 | miR-20b | CAAAGUGCUCAUAGUGCAGGUAG |
| SEQ ID NO. 42 | miR-93 | CAAAGUGCUGUUCGUGCAGGUAG |
| SEQ ID NO. 43 | miR-17 | CAAAGUGCUUACAGUGCAGGUAG |
| SEQ ID NO. 44 | miR-291a | CAUCAAAGUGGAGGCCCUCUCU |
| SEQ ID NO. 45 | miR-291b-5p | GAUCAAAGUGGAGGCCCUCUCC |
| SEQ ID NO. 46 | miR294 | AAAGUGCUUCCCUUUUGUGUGU |
| SEQ ID NO. 47 | miR-295 | AAAGUGCUACUACUUUUGAGUCU |
| SEQ ID NO. 48 | miR-302a | UAAGUGCUUCCAUGUUUUGGUGA |
| SEQ ID NO. 49 | miR-302b | UAAGUGCUUCCAUGUUUUAGUAG |
| SEQ ID NO. 50 | miR302c | UAAGUGCUUCCAUGUUUCAGUGG |
| SEQ ID NO. 51 | miR-302d | UAAGUGCUUCCAUGUUUGAGUGU |
| SEQ ID NO. 52 | miR-25 | CAUUGCACUUGUCUCGGUCUGA |
| SEQ ID NO. 53 | miR-32 | UAUUGCACAUUACUAAGUUGCA |
| SEQ ID NO. 54 | miR-92a-1 | UAUUGCACUUGUCCCGGCCUG |
| SEQ ID NO. 55 | miR-92a-2 | UAUUGCACUCGUCCCGGCCUCC |
| SEQ ID NO. 56 | miR-92b | UAUUGCACUCGUCCCGGCCUCC |
| SEQ ID NO. 57 | miR-363 | AAUUGCACGGUAUCCAUCUGUA |
| SEQ ID NO. 58 | miR-367 | AAUUGCACUUUAGCAAUGGUGA |
| SEQ ID NO. 59 | miR-19a | UGUGCAAAUCUAUGCAAAACUGA |
| SEQ ID NO. 60 | miR-19b | UGUGCAAAUCCAUGCAAAACUGA |
| SEQ ID NO. 61 | miR-290-5p | ACUCAAACUAUGGGGGCACUUU |
| SEQ ID NO. 62 | miR-292 | ACUCAAACUGGGGGCUCUUUUG |
| SEQ ID NO. 63 | miR-200c | UAAUACUGCCGGGUAAUGAUGGA |
| SEQ ID NO. 64 | miR-20a | UAAAGUGCUUAUAGUGCAGGUAG |
| SEQ ID NO. 65 | miR-290-3p | AAAGUGCCGCCUAGUUUUAAGCC |
| SEQ ID NO. 66 | miR-18b | UAAGGUGCAUCUAGUGCUGUUAG |
| SEQ ID NO. 67 | miR-291b-3p | AAAGUGCAUCCAUUUUGUUUGU |
| SEQ ID NO. 68 | miR-293 | AGUGCCGCAGAGUUUGUAGUGU |
| SEQ ID NO. 69 | miR-369-5p | AGAUCGACCGUGUUAUAUUCGC |

Measurement of pulse oximetry. The MouseOx Pulse-oximeter (Starr Life Sciences, Oakmont Pa.) was used to measure blood oxygen saturation (SpO2) in Sp infected mice. Mice were anesthetized and neck-hairs were removed using electric trimmer before infection. For readings, the oximeter clip was placed on the neck and percent SpO2 was measured each second over several minutes; data shown is the average of SpO2 readings recorded over 3-5 minutes per mouse.

Pulmonary function testing. Mice were anesthetized (4% isofluorane), tracheostomized (18 g), placed on the flexiVent system (SCIREQ, Montreal, QC, Canada), and ventilated with a tidal volume (7 mL/kg), at a frequency of 150 breaths/min and a positive end expiratory pressure of 3 cm H₂O. Anesthesia was titrated between 2-4% isofluorane to prevent spontaneous breathing; then, all pulmonary function measurements were performed at the same level of anesthesia and repeated in triplicate. After each measurement, the lung was conditioned to total lung capacity (ie. 30 cm H₂O). Whole lung dynamic respiratory mechanics were measured including pulmonary compliance and resistance determined by fitting the linear single-compartment model using a multiple linear regression, followed by the forced oscillation technique. In addition, negative pressure forced expirations were performed using the forced expiration extension for mice of the flexivent system to measure forced expiratory volumes (FEV) at specific time points during expiration (ie. FEV 0.1 sec), forced vital capacity (FVC) and subsequent for calculations of the ratio of forced expiratory volumes to forced vital capacity (ie. FEV 0.1 sec/FVC), as previously described. Total time of measurement was <15 min. All data were analyzed using FlexiVent software (version 7.5, service pack 4).

Quantification of total proteins and LDH activity in BAL. BAL was collected and centrifuged at 1650 rpm for 5 min at 4° C., supernatant was stored at −80° C. till detection. Total protein concentration was measured using Bradford protein assay kit (Bio-rad), LDH activity was assessed using the enzymatic detection of the CytoTox 96 non-radioactive cytotoxicity assays (Promega) according to the manufacturer's protocol and read on a PerkinElmer plate reader. 3-5 individual mice were measured and pooled together at each time points.

Epithelial cell isolation and flow cytometry. Lung epithelial cells were isolated as previously described. For FACS analysis, single-cell preparations were incubated for 30-45 min at 4° C. with the following primary antibodies: EpCAM (G8.8, eBioscience) and Streptavidin, R-Phycoerythrin Conjugate (SAPE), CD31 (1:500; ebioscience, 390), CD45 (1:500; BioLegend, 30-F11). Cleaved Caspase-3 (1:400, 5A1E, Cell Signaling Technology) and APC-conjugated secondary antibody. Incubations were done in PBS (without phenol red) plus 2% FBS, sample were collected on BD FACSCanto Flow Cytometer and analyzed using FlowJo.

miRNA in situ hybridization. In situ hybridizations were performed in 6-μm paraffin-embedded lung sections. Sections were deparaffinized, rehydrated in graded ethanols and pretreated with 10 μg/ml of proteinase K (Roche) for 10 minutes at room temperature. After protease digestion, the digoxigenin-labeled LNA miR-302c antisense probe or LNA-scrambled control probe (Exiqon) were hybridized to the slides in a humidified chamber at 60° C. overnight at a concentration of 20 nM in the hybridization buffer of 5×SSC, 50% formamide, 0.1% Tween-20, 500 μg/ml yeast RNA, 9.2 mM citric acid. Posthybridization washes were three times for 30 min each at 60° C. in 2×SSC 2×SSC, 50% formamide; five times 5 min each at 25° C. in PBS, 0.05% Tween-20 (PBST). Slides were blocked with 2% normal goat serum, 2 mg/ml BSA in PBST for 1 hour at 25° C., and incubated with antidigoxigenin alkaline phosphatase-conjugated antibody (Roche). Slides were rinsed in PBST and developed with nitro blue tetrazolium/5-bromo-4-chloro-3-indolyl-phosphate (NBT/BCIP) (Roche) to yield a blue-purple precipitate. Sections were mounted for viewing.

Statistical Analyses. Data are presented as mean±standard error of the mean (SEM). Unpaired, one-tailed, Student's t-test was used to calculate statistical significance between two groups and a one-way analysis of variance (ANOVA) for multiple group comparison followed by Bonferroni correction unless stated otherwise. All statistical tests were performed using Prism software (GraphPad Software). Data shown are means±SEM. P values are depicted as follows: * P<0.05; ** P<0.01; *** P<0.001. Results with P>0.05 were considered not significant (n.s.).

### Example 1: AEC Damage and Regeneration in Sp-Infected Mouse Lung

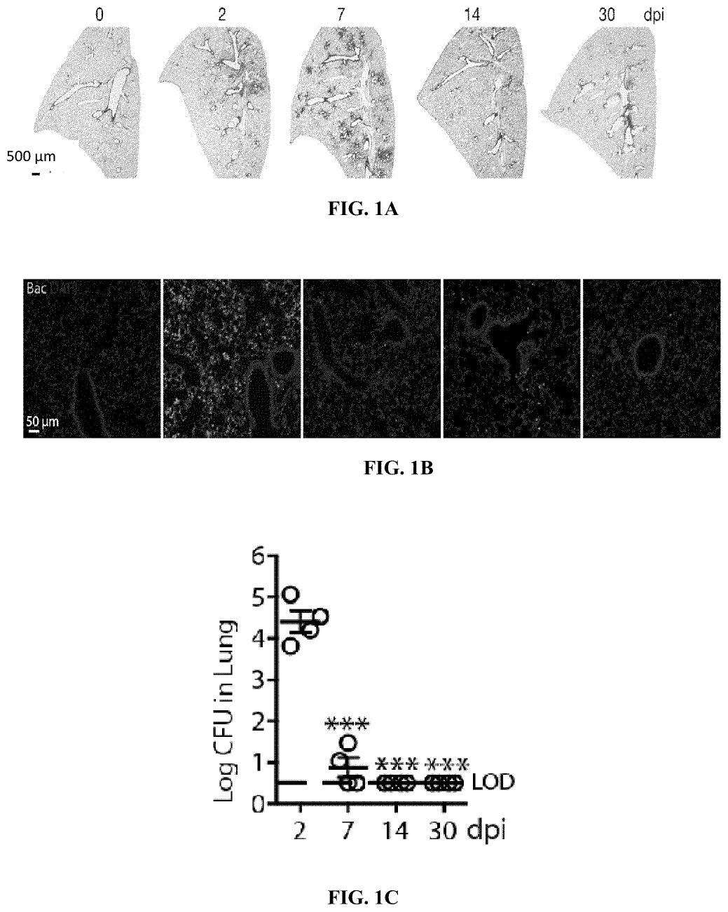

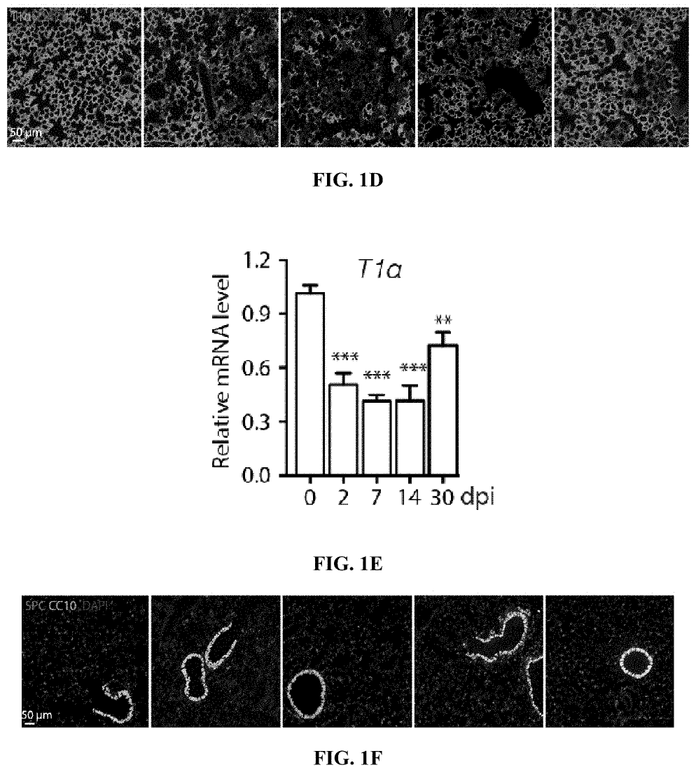

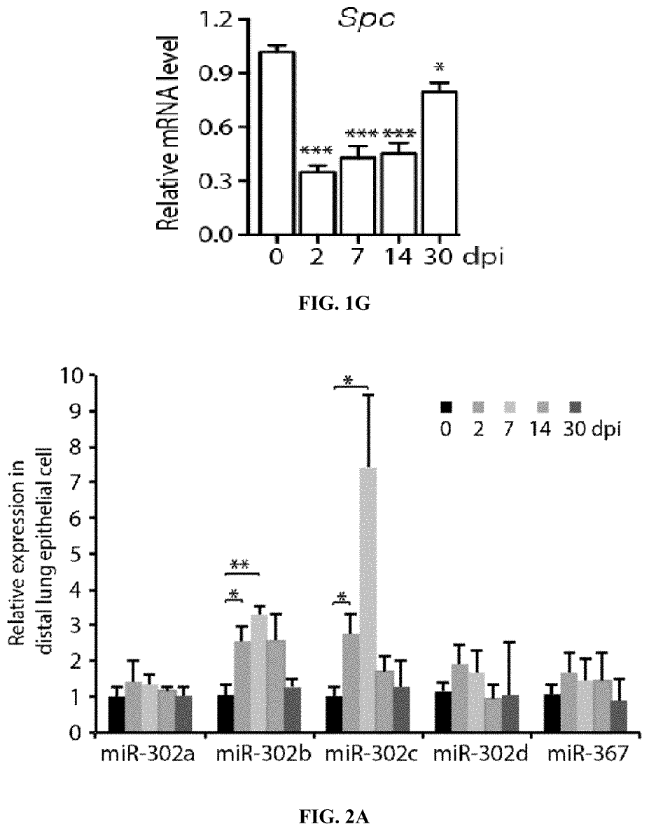

C57BL/6 mice were infected intranasally (i.n.) under anesthesia with ˜5×10\hat{ }6 colony-forming units (CFU) of the Sp TIGR4 strain (SpT4), resulting in direct infection of the lower respiratory tract and acute bacterial pneumonia with ˜40% mortality rate. Histology of lung sections of surviving mice showed numerous foci of inflammatory lesions starting at 2 days post-infection (dpi), peaking at 7 dpi, and returning to normal state by 30 dpi (FIG. 1A). Bacterial loads in the lung were determined by CFU plating and visualized by immunostaining of lung sections with antibodies specific to the type 4 capsule on the surface of SpT4 (FIG. 1B and FIG. 1C). Both CFU plating and immunostaining showed high levels of bacteria in the lung at 2 dpi that were cleared by 7 dpi, while immunostaining showed that most bacteria were localized to the alveolar spaces. Thus, this infection model recapitulates pathological and clinical features of severe acute pneumonia with extensive inflammation in the lung and infected mice either succumbing to infection or clearing bacteria.

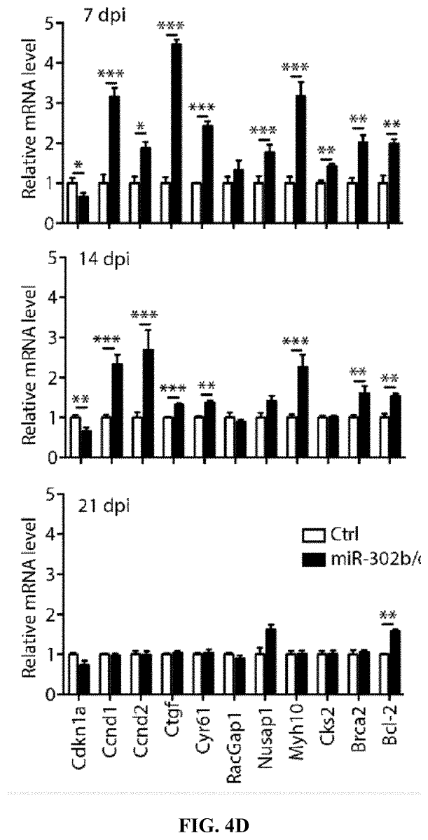

Lung alveoli are primarily composed of alveolar epithelial type I cells (AECI) that mediate the key function of gas exchange, and alveolar epithelial type II cells (AECII) that produce anti-microbial peptides and surfactant proteins and lipids for reducing alveolar surface tension. Flat-shaped AECI cells cover more than 90% of the alveolar surface and can be visualized by staining for the cell type specific markers T1α. Cuboidal-shaped AECII interdigitate between AECI and can be visualized by staining for the cell type specific markers SPC. Substantial destruction of AECI and AECII was observed after SpT4 infection, as evidenced by significant loss of immunostaining of the cell type specific markers for AECI (T1α) and AECII (SPC) at both mRNA and protein levels (FIGS. 1D-1G). Kinetics of AECI and AECII loss and regeneration were similar, with dramatic lost at 2 and 7 dpi, and recovery starting after 7 dpi, as visualized by immunostaining of T1α (AECI) and SPC (AECII) (FIGS. 1D and 1F). Similarly, gene expression analyses of cell-type specific markers by qRT-PCR for mRNA from the lung tissue showed decreased levels of T1α and SPC at 2, 7 and 14 dpi (FIGS. 1E and 1G). By 30 dpi, there was substantial recovery of AECI and AECII cells, though T1α and SPC mRNA levels were still reduced compared to those before infection (FIGS. 1E and 1G). In contrast to AEC, epithelial cells in the bronchiolar regions, such as Club cells (CC10), were unaffected (FIG. 1F), nor were other cells in the lung, including basal cells, ciliated cells, smooth muscle and fibroblasts, and vascular endothelial cells at 7 dpi (FIG. 5A). Modest levels of intra-alveolar fibrotic lesions were observed, as characterized by collagen fiber deposition and inflammatory cell accumulation in alveolar spaces, which subsequently resolved between 7-14 dpi (FIG. 5B). These results showed that mice with SpT4 infection exhibited extensive lung parenchyma injuries that impaired alveolar architecture with specific damage to AECI and AECII, rather than broad injury to all cell types. The regeneration and repair processes were slow and took more than 30 days for full recovery.

### Example 2: miRNA-302 Expression is Elevated in AEC after Sp Infection

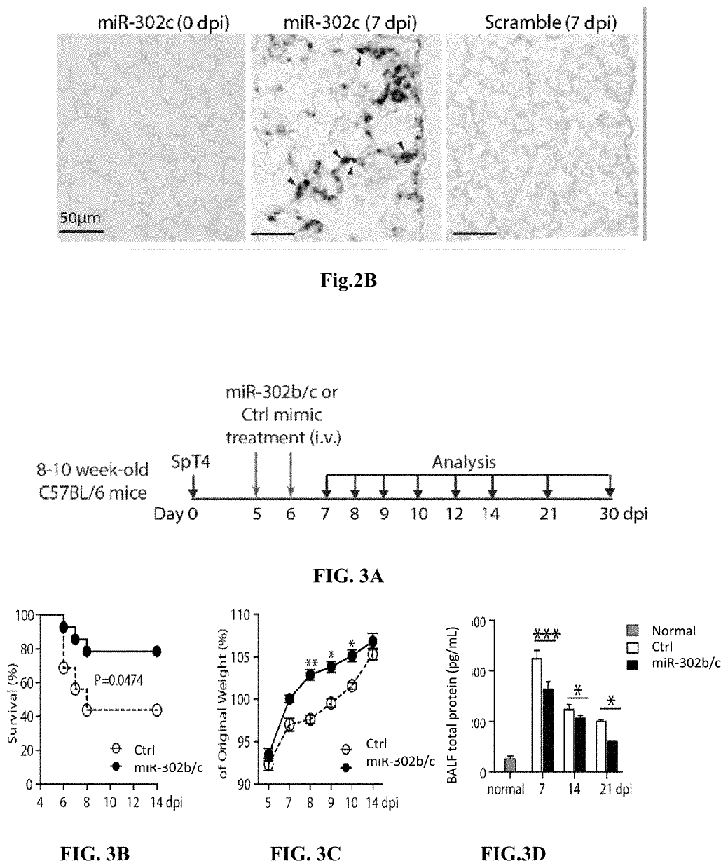

It is known that regulatory pathways important for tissue growth and differentiation during embryonic development can be reactivated in the process of regeneration to promote tissue repair. The microRNA cluster miR-302-367 is important for lung epithelial progenitor cell proliferation during embryonic development. Hence, a potential role of miR-302-367 was explored by asking if their expression is reactivated in the lung epithelium following injuries caused by bacterial pneumonia. While the expression of miR-302-367 was undetectable in the normal adult mouse lung epithelium, two members (miR-302b and miR-302c) of the miR-302-367 cluster were induced by SpT4 infection at 2 dpi, peaked at 7 dpi, and returned to the basal level by 30 dpi (FIG. 2A). Cells expressing miR-302c were evident in the alveolar epithelium by in situ hybridization at 7 dpi, but not before infection (FIG. 2B). Together, these data indicated that expression of miR-302b and miR-302c were reactivead in the adult lung epithelium after SpT4 infection, suggesting a potential role of miR-302b/c during tissue regeneration/repair in response to SpT4 infection-induced lung injury.

### Example 3: miRNA-302 mimic Treatment Improves Lung Function, Host Recovery and AEC regeneration in Sp-Infected Mice

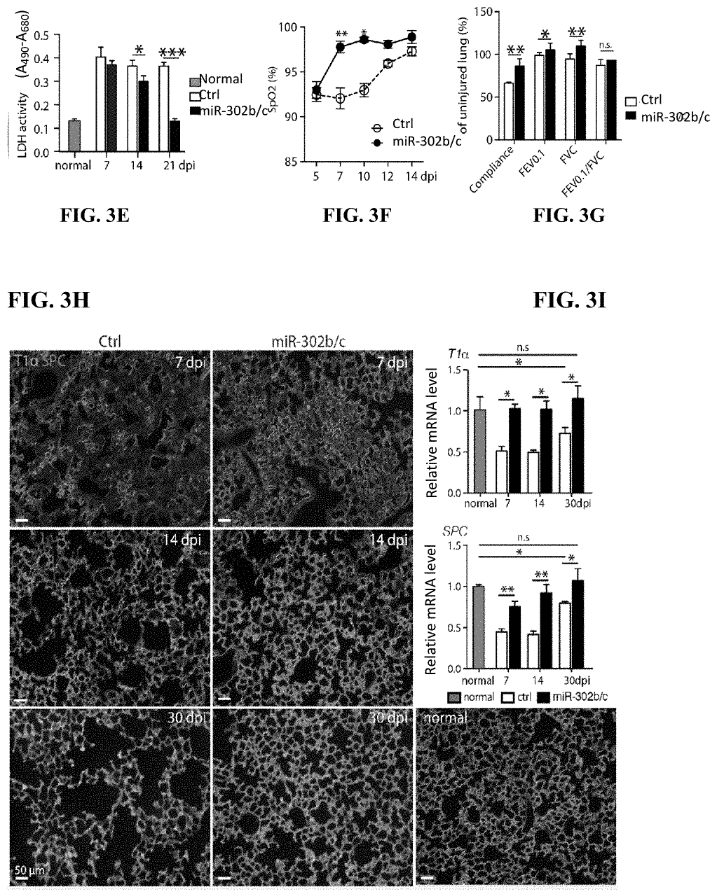

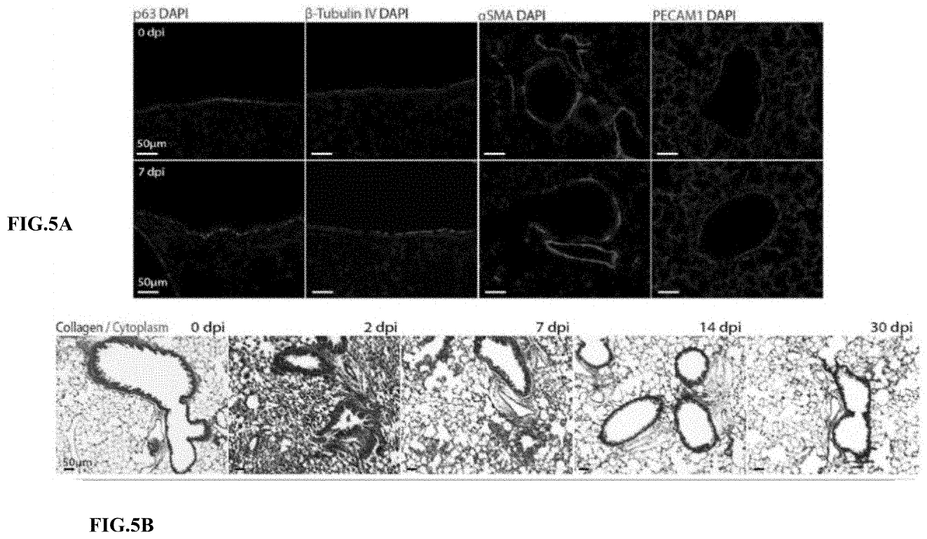

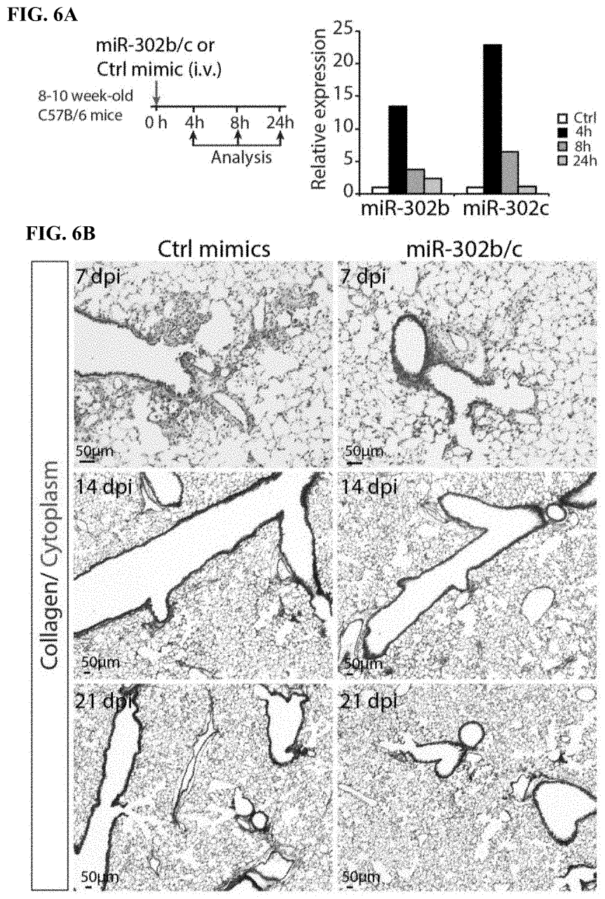

The role of miR-302b/c was tested using in vivo administration of miRNA mimics with the goal of developing novel therapeutics to improve recovery from bacterial pneumonia. To determine whether intravenous administration of miR-302b/c mimics led to accumulation of these miRNAs in the lung, lung tissues and observed miR-302b/c levels peaked at 4 hours and returned to baseline 24 hours after injection was examined (FIG. 6A). The SpT4-infected mice were then treated at 5 and 6 dpi with miR-302b/c mimics (miR-302b/c) or negative control mimics (Ctrl) by tail-vein injections (FIG. 3A). Treatment with miR-302b/c increased survival following infection compared to untreated, infected mice (FIG. 3B), and surviving treated mice regained body weight more rapidly than surviving untreated controls (FIG. 3C). Mice treated with miR-302b/c had lower levels of total protein and lactate dehydrogenase (LDH) activities in their bronchoalveolar lavage fluid (BALF) relative to the Ctrl group (FIGS. 3D and 3E), indicating less damage to lung tissue integrity in miR-302b/c-treated mice. Assessment of gas exchange by pulse oximetry (SpO2) showed accelerated recovery of oxygenation in the miR-302b/c-compared to Ctrl-treated group (FIG. 3F). Analysis of lung mechanics by flexiVent revealed significant improvements in compliance, forced expiratory volume (FEV0.1) and forced vital capacity (FEV) in miR-302b/c-treated animals compared to Ctrl (FIG. 3G). The FEV0.1/FVC ratio was not significantly affected, consistent with only modest levels of lung fibrotic lesion formation induced by SpT4 infection that was not affected by miR-302b/c treatment (FIG. 6B). Collectively, these data show that treatment with miR-302b/c mimics improved pulmonary function and recovery from bacterial pneumonia.

The effect of miR-302 mimic treatment on the process of lung AEC regeneration was evaluated. Analyses of lung sections by immunostaining (FIG. 3H) and qRT-PCR (FIG. 3I) of the T1α and SPC markers showed that miR-302b/c mimic-treated mice had a significant increase of AECI (T1α) and AECII (SPC) at 7, 14 and 30 dpi compared to the Ctrl group. By 30 dpi, the Ctrl mice still had lower expression of T1α and SPC markers compared to uninfected controls. In striking contrast, miR-302b/c mimic-treated mice fully recovered as there were no significant differences in expression of T1α and SPC markers between miR-302b/c mimic-treated and uninfected mice (FIG. 3I). Thus, therapeutic treatment with miR-302b/c mimics at 5 and 6 days post SpT4 infection promoted regeneration of AEC and repair of damaged alveolar epithelium, resulting in improved pulmonary function, lower mortality rate, and faster recovery of surviving animals from pneumonia.

### Example 4: miRNA-302 Mimic Treatment Increases AEC Proliferation In Vivo

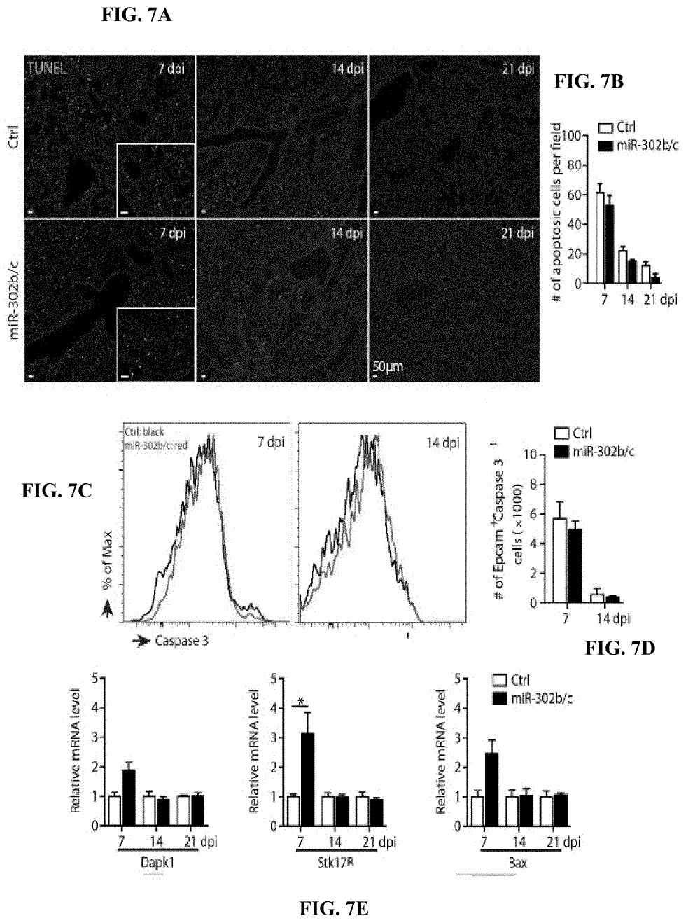

To study the mechanisms by which miR-302 mimic treatment might help recovery at cellular and molecular levels, its effect on apoptosis and proliferation of lung epithelial cells and on expression of genes associated with these processes, was examined. TUNEL staining of lung sections showed no significant difference in the number of apoptotic cells between miR-302b/c and Ctrl groups at 7, 14, 21 dpi (FIGS. 7A and 7B). Similarly, flow cytometry detecting cleaved caspase 3 from EpCAM⁺ epithelial cells showed no significant difference (FIGS. 7C and 7D). Furthermore, expression of apoptosis-associated genes (Dapk1, Stk17B, Bax) in lung epithelial cells as determined by qRT-PCR was not affected at 7, 14, and 21 dpi, except increased level of Stk17B at 7 dpi in miR-302b/c-treated mice (FIG. 7E). These results indicate that inhibition of apoptosis was unlikely to explain the effect of miR-302b/c mimics in reducing tissue injury during bacterial pneumonia.

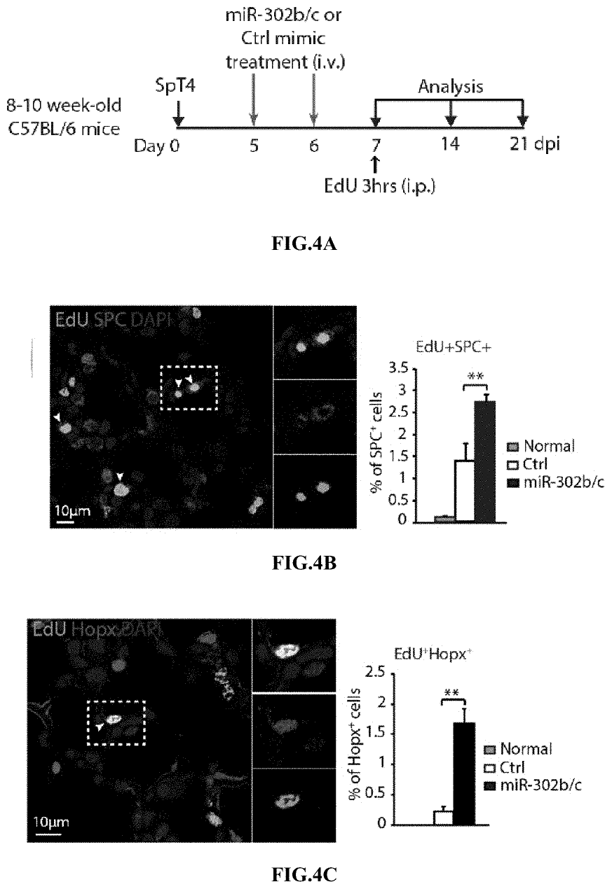

An alternative mechanism of miR-302b/c mimic treatment was stimulating proliferation of local progenitor cells and, thereby, enhancing regeneration of AEC, tissue repair and recovery. To examine cell proliferation in vivo following bacterial pneumonia, SpT4-infected mice, either treated with miR-302b/c or Ctrl mimic at 5 and 6 dpi, were pulsed with EdU for 3 hr at 7 dpi (FIG. 4A). Proliferating epithelial cells were quantified by visualizing EdU labeled cells co-immunostained with markers of AECI (Hopx) and AECII (SPC) (Barkauskas, et al (2013), J Clin Invest, 123(7), 3025-3036. Jain, R., et al., (2015). Nat Commun, 6, 6727.). There was a significant increase in AECI and AECII proliferation detected in the miR-302b/c-compared to the Ctrl-treated group (FIGS. 4B and 4C). Gene expression analyses using qRT-PCR showed that lung epithelium of miR-302b/c-treated mice had increased expression of genes associated with positive regulation of cell proliferation, including Ccnd1, Ccnd2, Ctgf, Cyr61, Nusap1, Myh10, Cks2, Brca2, compared with Ctrl-treated mice (FIG. 4D). In addition, expression of Cdknla, a cell cycle inhibitor gene, was reduced by miR-302b/c treatment (FIG. 4D). These results show that local epithelial cell proliferation, especially AECI and AECII, accounted for the accelerated repair in lung alveoli after miR-302b/c treatment following acute Sp-induced lung injury.

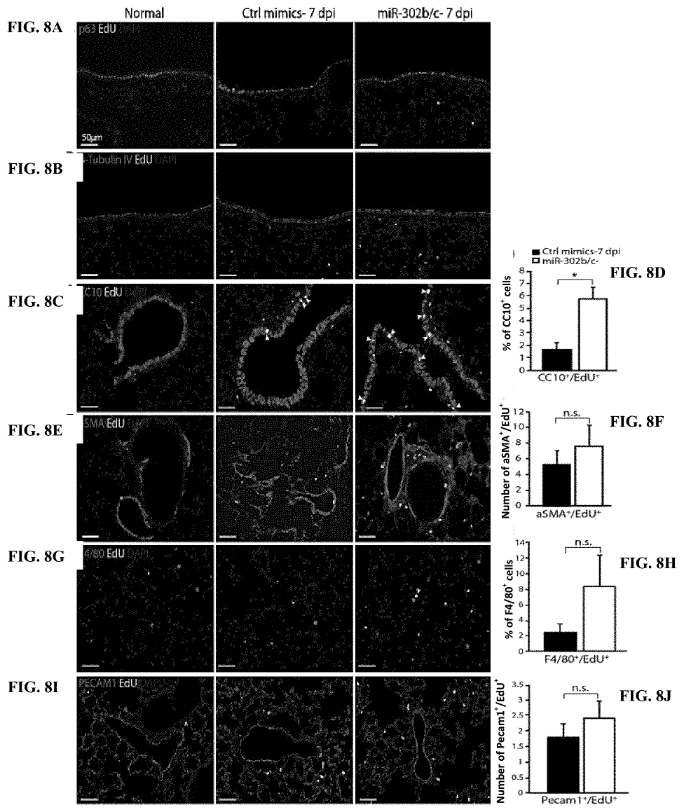

To determine possible effects of miR-302b/c treatment on other cells in the lung, cell proliferation was assessed by co-immunostaining of various cell-type specific markers and incorporation of EdU among different cell populations. No proliferation was detected in bronchial basal cells (p63) or ciliated cells (β-tubulin IV) in either miR-302b/c or Ctrl groups (FIGS. 8A and 8B). On the other hand proliferating bronchiolar Club cells (CC10), smooth muscle cells (αSMA), vascular endothelial cells (PECAM1), and macrophages (F4/80) at 7 dpi in both groups were observed (FIGS. 8C-8J). Further quantification revealed 3.4-fold more proliferating bronchiolar Club cells in the miR-302b/c-treated group (FIG. 8D), while proliferation of other three cell populations was not significantly different between the miR-302b/c and Ctrl groups (FIGS. 8F, 8H, 8J). Moreover, no differences were found in lung fibrotic lesion formation and resolution at 7, 14, 21 dpi between the miR-302b/c and Ctrl groups (FIG. 6B). These data indicate that miR-302 mimic treatment had a minimal effect on proliferation of cell types other than bronchiolar and alveolar epithelial cells in the lung.

### Example 5

The examples presented herein establish that bacterial pneumonia causes extensive damage to AEC in lung parenchyma and induces transient expression of miRNA-302 in the lung epithelium. Administration of miR-302 mimics to SpT4-infected mice improved AEC regeneration and lung function, and enhanced mouse recovery and survival. These results provide the first example of a signaling pathway important in embryogenesis that can be reactivated and exploited for regenerative lung therapy following microbial infection.

miRNA mimics are double-stranded RNA molecules intended to “mimic” native miRNAs; they have been used successfully to augment the function of endogenous microRNA in mouse models and are being tested in clinical trials for cancer treatment. It is shown in this study that a miRNA mimic approach can be used as a novel treatment of microbial infection by accelerating the proliferation of lung progenitor cells and regeneration of AEC to repair lung injury following bacterial pneumonia. These results suggest that the adult lung is capable of initiating a regenerative response after microbial infection to repair tissue injury by utilizing pathways typically expressed during embryogenesis and fetal lung development. However, the natural regenerative process is slow (not fully recovered after 30 days), leaving the host vulnerable to external insults and infections. Further, the transient expression of miR-302 in alveolar epithelium following bacterial pneumonia that is concomitant with regeneration of AEC and recovery of lung functions, suggest up-regulation of miR-302 may play a role in the regenerative process. Given the slow process of natural regeneration, it was reasoned that addition of exogenous miR-302 by miRNA mimics can increase proliferation of lung progenitor cells and accelerate the repair of lung injury and functions during bacterial pneumonia. Like other microRNAs, members of the miR-302 family engage a broad collection of mRNA targets, and the major targets of the miR-302 family are genes involved in cell cycle, proliferation and apoptosis. Indeed, the results show that miR-302 mimics resulted in up-regulation of genes associated with promoting cell proliferation and repression of pro-apoptotic genes, which likely contributes to enhanced proliferation of AECI and AECII observed in miR-302 mimic-treated mice. AECI and AECII are known local progenitor cells in lung alveoli. Injuries models using either chemical or mechanical insults show that AECII and AECI increased their proliferation to replace the lost alveolar epithelial cells and contribute to the repair/regeneration of alveolar epithelium. The miR-302 targets the cell cycle inhibitor (Cdkn1a) and expression of miR-302 is essential for the proliferation of lung epithelial progenitor cells during embryonic development. miR-302b/c mimic treatment led to decreased expression of Cdkn1a in lung epithelium (FIG. 4D). Together, these data suggest that the mechanisms by which miR-302b/c mimics promote mouse lung repair/regeneration and host recovery from bacterial pneumonia is by regulating expression of genes that promote transient activation of AECII and AECI proliferation.

Efficient in vivo delivery of miRNA mimics to targeted cells is the key to the success of this approach. The i.v. delivery results in accumulation of miR-302b/c mimics in the lung. In fact, delivery by i.v. is an efficient mean to deliver drugs to the lung because the entire right side of the heart is dedicated to pump blood exclusively into the lung. More importantly, bacterial pneumonia causes substantial damage to the integrity of lung epithelium, which allowing efficient penetration of i.v. delivered drug into lung epithelial cells. Systematic administration of miRNA mimics resulted in rapid therapeutic effect shortly after the second dose, including increased proliferation of AECI and AECII cells and improved lung function (FIGS. 3A-3I and FIGS. 4A-4D). One downside of this systematic delivery approach is accumulation and possible off-site effects in other organs. The miR-302 mimics delivered by i.v. injection did not cause adverse effect in other organs including heart, liver, and intestine by H&E histology. Furthermore, bacterial pneumonia is an acute infection mostly limited to the lung. In the current model, no bacteria or tissue injuries were detected in other organs, and the resulting immune responses were localized to the lung mucosa with minimal responses in the spleen. Thus, it is expected that miR-302b/c mimics have most effect in the lungs of SpT4-infected mice and minimal adverse effects in other organs.

In conclusion, the results presented herein open up a new frontier in developing therapies for treating microbial infection by regenerative medicine. Although this regenerative approach does not curtail microbial growth per se, it reduces suffering and shortens recovery time by fostering tissue repair and, thus, can be used in conjunction with antimicrobial therapy to improve patient outcomes from serious infections. In addition to treating acute pneumonia, this approach may provide long-term benefit of reducing severe chronic pathological conditions, such as COPD, as repeated lung infections/injuries and defects in tissue regeneration/repair have been implicated in these devastating conditions.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating a lung injury in a subject in need thereof, the method comprising administering to the subject a therapeutically effective amount of at least one miR-302 mimic.

2. The method of claim 1, wherein the at least one miR-302 mimic comprises miR-302b mimic or miR-302c mimic.

3. The method of claim 1, wherein administering the at least one miR-302 mimic promotes regeneration of alveolar epithelial cells I (AECI) and alveolar epithelial cells II (AEC II) in a lung of the subject.

4. The method of claim 1, wherein administering the at least one miR-302 mimic results in upregulation of expression of at least one cell proliferation gene in a lung of the subject.

5. The method of claim 4, wherein the at least one cell proliferation gene is selected from the group consisting of Ccnd1, Ccnd2, Ctgf, Cyr61, Nusap1, Myh10, Cks2 and Brca2.

6. The method of claim 1, wherein administering the at least one miR-302 mimic results in downregulation of expression of Cdkn1a gene.

7. The method of claim 1, wherein the lung injury is caused by a bacterial infection.

8. The method of claim 7, wherein the bacterial infection is bacterial pneumonia.

9. The method of claim 1, wherein the at least one miR-302 mimic is administered intravenously.

10. The method of claim 1, wherein the at least one miR-302 mimic further comprises a pharmaceutically acceptable carrier or adjuvant.

11. The method of claim 1, wherein the at least one miR-302 mimic is mammalian.

12. The method of claim 11, wherein the at least one miR-302 mimic is human.

13. The method of claim 1, wherein the at least one miR-302 mimic is engineered.

14. The method of claim 1, wherein the subject is mammal.

15. The method of claim 14, wherein the mammal is human.

16. A method of regeneration of alveolar epithelial cells I (AEC) and alveolar epithelial cells II (AECII) in lungs of a subject, the method comprising administering to the subject a composition comprising a therapeutically effective amount of at least one miR-302 mimic.

17. The method of claim 16, wherein the composition further comprises a pharmaceutically acceptable carrier or adjuvant.

18. The method of claim 16, wherein the at least one miR-302 mimic comprises a miR-302b mimic or miR-302c mimic.

19. A kit comprising a composition comprising at least one miR-302 mimic, and an instructional material for use thereof, wherein the instructional material comprises instructions for treating a lung injury in a subject, wherein the treating includes administering the at least one miR-302 mimic to the subject.

20. The kit of claim 19, wherein the at least one miR-302 mimic comprises a miR-302b mimic or miR-302c mimic.


SEQUENCE LISTING (69 sequences)

SEQ ID NO: 1 - DNA, 20 bp, Artificial Sequence
  Primer
ggtgcccccaggtatagaag

SEQ ID NO: 2 - DNA, 20 bp, Artificial Sequence
  Primer
acgctctctctgcgttggta

SEQ ID NO: 3 - DNA, 21 bp, Artificial Sequence
  Primer
accctgtgtggagagctacca

SEQ ID NO: 4 - DNA, 19 bp, Artificial Sequence
  Primer
tttgcggagggtctttcct

SEQ ID NO: 5 - DNA, 26 bp, Artificial Sequence
  Primer
ataccctcccacaagagaccaggata

SEQ ID NO: 6 - DNA, 24 bp, Artificial Sequence
  Primer
acacagggcagtgacaaggcttta

SEQ ID NO: 7 - DNA, 20 bp, Artificial Sequence
  Primer
gctgaacatggagctgactt

SEQ ID NO: 8 - DNA, 22 bp, Artificial Sequence
  Primer
caaggagggtcttgatgacttc

SEQ ID NO: 9 - DNA, 22 bp, Artificial Sequence
  Primer
aatctgcatgaggtctacgaaa

SEQ ID NO: 10 - DNA, 21 bp, Artificial Sequence
  Primer
tcggctaactcaggtaaacac

SEQ ID NO: 11 - DNA, 21 bp, Artificial Sequence
  Primer
gtggttgccctcttctacttt

SEQ ID NO: 12 - DNA, 20 bp, Artificial Sequence
  Primer
cagcccatgatggttctgat

SEQ ID NO: 13 - DNA, 24 bp, Artificial Sequence
  Primer
ttgtacaaggagccaggccaagat

SEQ ID NO: 14 - DNA, 24 bp, Artificial Sequence
  Primer
actaagtgctttgacacccacggt

SEQ ID NO: 15 - DNA, 20 bp, Artificial Sequence
  Primer
ttcctctccaaaatgccaga

SEQ ID NO: 16 - DNA, 20 bp, Artificial Sequence
  Primer
agggtgggttggaaatgaac

SEQ ID NO: 17 - DNA, 20 bp, Artificial Sequence
  Primer
gaacctggccgcagtcaccc

SEQ ID NO: 18 - DNA, 20 bp, Artificial Sequence
  Primer
cgacggcgggtacatggcaa

SEQ ID NO: 19 - DNA, 18 bp, Artificial Sequence
  Primer
aaccgcaagatcggagtg

SEQ ID NO: 20 - DNA, 19 bp, Artificial Sequence
  Primer
tgctttggaaggactcacc

SEQ ID NO: 21 - DNA, 20 bp, Artificial Sequence
  Primer
tgagttaatcgcaattggaa

SEQ ID NO: 22 - DNA, 21 bp, Artificial Sequence
  Primer
gtggtctgaacgatgcatttc

SEQ ID NO: 23 - DNA, 22 bp, Artificial Sequence
  Primer
cagatccagtgacaatgttcca

SEQ ID NO: 24 - DNA, 22 bp, Artificial Sequence
  Primer
tccaccatcatgaactgattcc

SEQ ID NO: 25 - DNA, 21 bp, Artificial Sequence
  Primer
gaggaggaagaagcacaagac

SEQ ID NO: 26 - DNA, 26 bp, Artificial Sequence
  Primer
ctactatcagttcctttcatctccaa

SEQ ID NO: 27 - DNA, 21 bp, Artificial Sequence
  Primer
ggaattcgagaggcagaacaa

SEQ ID NO: 28 - DNA, 20 bp, Artificial Sequence
  Primer
aaggctcgcttggatttctc

SEQ ID NO: 29 - DNA, 24 bp, Artificial Sequence
  Primer
cagagtctaggatgggttcattac

SEQ ID NO: 30 - DNA, 19 bp, Artificial Sequence
  Primer
tcccagctgcacttcattt

SEQ ID NO: 31 - DNA, 18 bp, Artificial Sequence
  Primer
atttgaacggcccagcat

SEQ ID NO: 32 - DNA, 22 bp, Artificial Sequence
  Primer
ggctggtaaacctggagtaaag

SEQ ID NO: 33 - DNA, 22 bp, Artificial Sequence
  Primer
gtggatgactgagtacctgaac

SEQ ID NO: 34 - DNA, 21 bp, Artificial Sequence
  Primer
gagacagccaggagaaatcaa

SEQ ID NO: 35 - DNA, 20 bp, Artificial Sequence
  Primer
tgcaccaccaactgcttagc

SEQ ID NO: 36 - DNA, 21 bp, Artificial Sequence
  Primer
ggcatggactgtggtcatgag

SEQ ID NO: 37 - RNA, 23 nt, Artificial Sequence
  Oligo
uaagugcuuccauguuuuaguag

SEQ ID NO: 38 - RNA, 22 nt, Artificial Sequence
  Oligo
aagugcuuccauguuucagugg

SEQ ID NO: 39 - RNA, 23 nt, Artificial Sequence
  miRNA mimic
caaagugcuaacagugcagguag

SEQ ID NO: 40 - RNA, 21 nt, Artificial Sequence
  miRNA mimic
uaaagugcugacagugcagau

SEQ ID NO: 41 - RNA, 23 nt, Artificial Sequence
  miRNA mimic
caaagugcucauagugcagguag

SEQ ID NO: 42 - RNA, 23 nt, Artificial Sequence
  miRNA mimic
caaagugcuguucgugcagguag

SEQ ID NO: 43 - RNA, 23 nt, Artificial Sequence
  miRNA mimic
caaagugcuuacagugcagguag

SEQ ID NO: 44 - RNA, 22 nt, Artificial Sequence
  miRNA mimic
caucaaaguggaggcccucucu

SEQ ID NO: 45 - RNA, 22 nt, Artificial Sequence
  miRNA mimic
gaucaaaguggaggcccucucc

SEQ ID NO: 46 - RNA, 22 nt, Artificial Sequence
  miRNA mimic
aaagugcuucccuuuugugugu

SEQ ID NO: 47 - RNA, 23 nt, Artificial Sequence
  miRNA mimic
aaagugcuacuacuuuugagucu

SEQ ID NO: 48 - RNA, 23 nt, Artificial Sequence
  miRNA mimic
uaagugcuuccauguuuugguga

SEQ ID NO: 49 - RNA, 23 nt, Artificial Sequence
  miRNA mimic
uaagugcuuccauguuuuaguag

SEQ ID NO: 50 - RNA, 23 nt, Artificial Sequence
  miRNA mimic
uaagugcuuccauguuucagugg

SEQ ID NO: 51 - RNA, 23 nt, Artificial Sequence
  miRNA mimic
uaagugcuuccauguuugagugu

SEQ ID NO: 52 - RNA, 22 nt, Artificial Sequence
  miRNA mimic
cauugcacuugucucggucuga

SEQ ID NO: 53 - RNA, 22 nt, Artificial Sequence
  miRNA mimic
uauugcacauuacuaaguugca

SEQ ID NO: 54 - RNA, 21 nt, Artificial Sequence
  miRNA mimic
uauugcacuugucccggccug

SEQ ID NO: 55 - RNA, 22 nt, Artificial Sequence
  miRNA mimic
uauugcacucgucccggccucc

SEQ ID NO: 56 - RNA, 22 nt, Artificial Sequence
  miRNA mimic
uauugcacucgucccggccucc

SEQ ID NO: 57 - RNA, 22 nt, Artificial Sequence
  miRNA mimic
aauugcacgguauccaucugua

SEQ ID NO: 58 - RNA, 22 nt, Artificial Sequence
  miRNA mimic
aauugcacuuuagcaaugguga

SEQ ID NO: 59 - RNA, 23 nt, Artificial Sequence
  miRNA mimic
ugugcaaaucuaugcaaaacuga

SEQ ID NO: 60 - RNA, 23 nt, Artificial Sequence
  miRNA mimic
ugugcaaauccaugcaaaacuga

SEQ ID NO: 61 - RNA, 22 nt, Artificial Sequence
  miRNA mimic
acucaaacuaugggggcacuuu

SEQ ID NO: 62 - RNA, 22 nt, Artificial Sequence
  miRNA mimic
acucaaacugggggcucuuuug

SEQ ID NO: 63 - RNA, 23 nt, Artificial Sequence
  miRNA mimic
uaauacugccggguaaugaugga

SEQ ID NO: 64 - RNA, 23 nt, Artificial Sequence
  miRNA mimic
uaaagugcuuauagugcagguag

SEQ ID NO: 65 - RNA, 23 nt, Artificial Sequence
  miRNA mimic
aaagugccgccuaguuuuaagcc

SEQ ID NO: 66 - RNA, 23 nt, Artificial Sequence
  miRNA mimic
uaaggugcaucuagugcuguuag

SEQ ID NO: 67 - RNA, 22 nt, Artificial Sequence
  miRNA mimic
aaagugcauccauuuuguuugu

SEQ ID NO: 68 - RNA, 22 nt, Artificial Sequence
  miRNA mimic
agugccgcagaguuuguagugu

SEQ ID NO: 69 - RNA, 22 nt, Artificial Sequence
  miRNA mimic
agaucgaccguguuauauucgc